(19) United States
(12) Patent Application Publication
(10) Pub. No.: US 2009/0269737 A1
(43) Pub. Date: Oct. 29, 2009

(54) Integrated non-homogeneous nucleic acid amplification and detection

(75) Inventor: Pia Ollikka, Littoinen (FI)

Correspondence Address: BUCHANAN, INGERSOLL & ROONEY PC, POST OFFICE BOX 1404, ALEXANDRIA, VA, 22313-1404, US

(73) Assignee: WALLAC OY, Turku (FI)

(21) Appl. No.: 11/630,790

(22) PCT Filed: Jun. 28, 2005
(86) PCT No.: PCT/FI05/50244

§ 371 Date: Jun. 12, 2007

Related U.S. Application Data
(60) Provisional application No. 60/583,362, filed on Jun. 29, 2004.

(30) Foreign Application Priority Data
Jun. 29, 2004 (FI) 20045247

(51) Int. Cl. C12Q 1/68

(52) U.S. Cl. 435/6

(57) ABSTRACT

The present invention relates to an integrated method of amplifying and analyzing target nucleic acids, in which immobilized or immobilizable oligonucleotide capture probes are provided and a nucleic acid containing sample to be analyzed is added together with a reagent mixture, which mixture contains all reagents needed for amplification and subsequent analysis of said target nucleic acids. In the method amplification of the target nucleic acids, hybridization of said amplified target nucleic acids to the capture probes and separating the hybrids formed from un-reacted components, as well as the detection and measuring of the amount of labeled, hybridized target nucleic acids by means of a detectable signal, is performed in one reaction chamber. Further provided are reagent mixtures and kits for use in such methods.

### CROSS REFERENCE TO RELATED APPLICATIONS

This application claims priority under 35 U.S.C. §119 to Finnish Application 20045247 filed in Finland on 29 Jun. 2004; priority under 35 U.S.C. §119(e) to U.S. Provisional application 60/583,362 filed on 29 Jun. 2004; and is a US national phase application of PCT/FI2005/050244 filed as an International Application on 28 Jun. 2005 designating the U.S., the entire contents of which are hereby incorporated by reference in their entireties.

### FIELD OF THE INVENTION

The present invention relates to an integrated non-homogeneous method of amplifying and analysing nucleic acids, and more particularly to a method of performing said amplification and analysing steps in one single container.

The methods of the present invention are easily automated and pro-vide minimized risks related to contamination due to minimal handling of the samples.

### BACKGROUND OF THE INVENTION

As part of a rapidly increasing wealth of genetic information many disease-associated SNP (single nucleotide polymorphism) markers have been identified. In multifactorial disorders, risk calculation may require analysis of multiple SNPs. The increased number of tests to be run raises a need for cost-effective operations based on integration and automation of the assay procedures, and the automation of the whole DNA analysis is an increasing task in many genetic laboratories. Although several attempts to perform DNA-analysis as High Throughput Assays has been described, the logistics of sample handling from sample preparation and pre-treatment to the ultimate analysis still requires manual handling and physical transportation of the samples. In addition, the need to physically transfer amplified DNA samples within the lab poses a serious contamination risk.

There are several automated stations for sample preparation avail-able on the market and robotics offer a solution for transferring samples to the amplification reaction chambers. One known solution for minimizing the handling of PCR products is the emergence of so called closed-tube assays. In closed-tube assays the PCR product is analyzed in the amplification tube by a homogeneous method as described in e.g., U.S. Pat. No. 5,210,015.

A disadvantage of homogeneous assay formats is that only a few, usually one to two genotypings, may be performed in the same tube. Genotyping of tens, hundreds or even thousands of genetic markers may be of interest in predisposition profiling in complex diseases as well as early diagnosis and perceive prognosis and individualized therapy in various areas including for example oncology and infectious diseases. If there are numerous targets to be analyzed, non-homogeneous assay formats are preferred, as they are more easily multiplexed. One example of such assay formats is DELFIA® hybridization, on arrays of beads or spots.

Array technologies are mainly used in gene expression profiling and SNP scoring but it is likely to become a standard tool in clinical diagnostics as well. In an array, the separate reaction points can be separated either by spatial resolution on a planar spot array, or in suspension by physically separating the reactions on differently coded beads. Typically, the arrays are detected after separating the unbound reactants by wash but optical separation by con-focal optics is also used.

Genotyping and DNA sequencing on arrays require that DNA is extracted from a biological source for amplification purposes and afterwards analysed on the array. Typically, the amplification step is performed in amplification tubes, whereafter the amplified sample is transferred to a hybridization vessel. The need of physical transfer of amplified DNA samples within the lab poses a serious contamination risk making automation challenging. There is a need to develop systems for multiplex nucleic acid analysis that are less prone to contamination.

PCR on array is the most suitable method for allele-specific amplification, but this is a technically demanding, highly multiplexed PCR, not a multiplexed hybridization. Solid-phase PCR using arrayed primers is described e.g., in International Patent Publication WO 96/26291. So far, attempts to integrate the system have suffered from inefficiency.

### BRIEF DESCRIPTION OF THE INVENTION

The present invention provides a solution to the known shortcomings of the prior art, such as minimal handling of the amplification product, thus minimizing the risk of contamination in heterogeneous assays.

An object of the present invention is thus to provide an integrated method of amplifying and analysing target nucleic acids, in which immobilized or immobilizable oligonucleotide capture probes are provided on a solid sup-port and a nucleic acid containing sample to be analyzed is added together with a reagent mixture, which mixture contains all reagents needed for amplification and subsequent analysis of said target nucleic acids. In the method according to the present invention the amplification of the target nucleic acids, capturing hybridization of said amplified target nucleic acids with the capture probes and separating the hybrids formed from un-reacted components, as well as the detection and measuring of the amount of labelled, hybridized tar-get nucleic acids by means of a detectable signal, is performed in one reaction chamber.

In a specific embodiment of the present invention there is provided a method, wherein the amplification step involves thermo cycling and the hybridization step is performed after the amplification by lowering the reaction temperature to a level allowing hybridization.

Further provided is a reaction mixture for use in a method according to the present invention, comprising all necessary reagents for performing the method, including oligonucleotide primers labelled with a thermo-stable first member of a biorecognition pair; dNTP's and/or NTP's; at least one polymerase; amplification and hybridization buffers; and a detectably labelled second member of the biorecognition pair of reagent (i). In a preferred embodiment said first member of a biorecognition pair is biotin and said second member of the biorecognition pair is thermo stable avidin or streptavidin.

Further provided is a kit for use in a method according to the pre-sent invention, comprising a reaction mixture according the present invention and pre-immobilized target specific oligonucleotide probes.

The objects of the invention are achieved by a method and an arrangement, which are characterized by what is stated in the independent claims. The preferred embodiments of the invention are disclosed in the de-pendent claims.

### BRIEF DESCRIPTION OF THE DRAWINGS

In the following the invention will be described in greater detail by means of preferred embodiments with reference to the attached drawings, in which

FIG. 1 is a schematic presentation of an integrated PCR and detection method according to the present invention;

FIG. 2 is a schematic presentation of a DELFIA® PCR assay according to the present invention;

FIG. 3 represents images comparing results of integrated methods according to the present invention;

FIG. 4 shows the result of a DELFIA® PCR assay;

FIG. 5 shows the result of a DELFIA® PCR assay on a saliva sample;

FIG. 6 shows the result of an experiment comparing conventional PCR in a tube (A), in a microtiter well (C) and an integrated method according to the present invention (E).

### DETAILED DESCRIPTION OF THE INVENTION

The present invention relates to an integrated, non-homogeneous method of amplifying and analysing nucleic acids, and more particularly to a method of performing said amplification and analysing steps in one single reaction chamber. Said method is easily automated and there is a minimized risk related to contamination due to minimal handling of the samples.

The method of the present invention comprises the following steps:

i) providing pre-immobilized or immobilizable oligonucleotide capture probes;
ii) providing a sample containing target nucleic acids and a reagent mixture, comprising necessary reagents for amplification and subsequent analysing;
iii) amplifying the target nucleic acids;
iv) hybridizing the amplified target nucleic acids to the capture probes;
v) separating the formed hybrids from un-reacted components by capture on a solid support;
vi) detecting and measuring the amount of hybridized target nucleic acids by means of a detectable signal.

“Oligonucleotide capture probes” according to the present invention are thermo-stable oligonucleotides, which are pre-immobilized onto a solid support matrix, preferably onto a wall of a reaction chamber for performing the integrated assay. The main requirement of the matrix is that it has to allow thermo-stable coupling of the probes. The capture probes need not be pre-immobilized, but may be provided in immobilizable form, so that they can be immobilized by the aid of a tag or other capturing moiety linked to the probe.

In one preferred embodiment the matrix is a microtitration plate, wherein the separate wells serve as the reaction chambers. Suitable plates are readily available, and may include amino, carboxyl, N-oxysuccinimide or otherwise functionalized heat stable polymers, such as polypropylene or poly-carbonate, silica or glass.

In another preferred embodiment of the present invention the solid support matrix may be provided in the form of beads. In such an embodiment said beads may constitute an array, as different specific capture probes may be immobilized to different sets of beads. Suitable matrices for use as beads include the thermo stable polymers listed above.

Examples of suitable microtitration plate are available, such as NU-CLEOLINK™ plate, provided by Nunc and DNA-BIND™ provided by Corning.

A “sample containing target nucleic acids” is meant to include any biological sample containing nucleic acids to be analysed, including bodily flu-ids, such as whole blood, saliva, sputum, urine, faecal, peritoneal and pleural fluids; lavations, such as bronchoalveolar, nasal, cervical and intestinal samples; aspiration or biopsy samples; cell cultures or microbial cultures. The biological samples may also be samples taken from food or environmental samples. In addition to complex biological samples, the method of the present invention is equally suitable for amplifying and analysing pre-treated samples, for example samples where DNA and/or RNA has already been extracted.

In a preferred embodiment of the present invention, the reagent mixture includes reagents for performing the amplification reaction as well as means for detecting the resulting hybridization products. The amplification reagents include specific oligonucleotide primer pairs, nucleotides and thermo-stable DNA or RNA polymerase. Amplification reagent mixtures are known in the art, and the man skilled in the art may is able to compose the mixture, depending on the type of amplification reaction used and on the sample to be analysed.

Detection means for use in a method according to the present invention include any labelling methods known in the art, including direct and indirect methods, for labelling and detecting nucleic acids. Any known label is suitable for use in the method according to the present invention provided the label is thermo stable to such a degree, that the label is not destroyed during the thermo cycling or other heated processes of amplification. Preferred labelling methods include direct labelling, such as chemical modification by fluorescent, luminescent or absorbing label structures or nano beads, as well as indirect labelling. Indirect labelling methods useful in the present invention include e.g., enzymatic labelling methods, use of secondary pre-labelled hybridization probes and labelling methods based on biorecognition pairs, such as biotin-avidin, biotin-streptavidin or other affinity-based pairing.

In one preferred embodiment of the present invention, the reagent mixture does not include the labelling agents. In this embodiment the labelling agents are added to the reaction chamber prior to step iv). This embodiment allows the use of non-thermo stable labels. Adding the label during at a later step of the process is possible, if the addition is performed in such a way that there is no risk of contamination, e.g., if the label is added through a sealing film or the like.

The term “amplification” is meant to include any method for amplifying nucleic acids known in the art, either thermal cycling methods such as polymerase chain reaction (PCR; U.S. Pat. No. 4,683,202), reverse transcriptase PCR, (U.S. Pat. No. 5,310,652), and ligase chain reaction (LCR; U.S. Pat. No. 5,185,243), and any variations thereof, or isothermal methods, such as Q-Beta replicase technology (U.S. Pat. No. 4,786,600), nucleic acid sequence based amplification (NASBA; U.S. Pat. No. 5,409,818), transcription mediated amplification (TMA; U.S. Pat. No. 5,399,491), strand displacement amplification (SDA; U.S. Pat. No. 5,455,166) and multiple displacement amplification (MDA; U.S. Pat. No. 6,124,120).

One preferred method of amplification is asymmetric PCR, de-scribed by Innis et al., PNAS 85(24), 1988: 9436-40, which generates single stranded amplification products. Other known amplification methods may also be useful in the integrated assay according to the present invention.

In the present method, the amplification as well as the hybridization and the subsequent detection are performed in the same reaction chamber. In a highly preferred embodiment of the present invention the hybridization step is achieved by lowering the reaction temperature after performing the amplification reaction. As the reagent mixture contains all necessary reagents, no additions or other handling steps are needed at this moment in addition to the change in reaction temperature.

The separation step is performed according to known methods depending on the type of reaction chamber and solid support matrix used. In a preferred embodiment of the present invention, where the reaction chamber is a microtitration well, the separation step may be performed as a standard washing procedure, where standard wash solutions are added and removed from the reaction chamber, for example as described in the manufacturer's brochure DELFIA® Celiac Disease Hybridization Assay.

In other embodiments using array of beads on microtitration plates provided with filter bottoms as reaction chambers, the separation step may be performed by adding wash solution and subsequently removing the wash solution and un-reacted components by filtration by pumping or pressure from the top as well as by suction from the bottom.

In a further preferred embodiment the separation is not performed by physical separation such as washing and/or filtration, but is based on an optical separation of formed and labelled hybrids from un-reacted components. In such an embodiment the detectable label is measured by confocal scanning or imaging, e.g. by two-photon excitation. Other suitable methods of detection and measurement are easily appreciated by those skilled in the art.

Preferred method of detection is using bead/spot array on transparent assay well bottom and having image analysis through the bottom focused to the surface allowing spot/bead image collection without the interference from the unreacted compounds in the supernatant. The image can be either regular fluorescence or time-resolved.

Another preferred method for the detection of the hybrids is confocal scanning of bead (suspension) through the assay lid or bottom using for example two-photon excitation to create the confocal field.

The integrated assay format according to the present invention is exemplified below using amino functionalized oligonucleotide probes coupled to a thermo stable functionalized microtitration well (NULCEOLINK™, Nunc) by a spot assayer BIOCHIP ARRAYER™, Packard). The oligonucletide modification is described by Hovinen and Hakala in Org. Lett. 2001, 3(16):2473-6, and relates to a modification of a reactive amino group to urasil. The coupling of the oligonucleotide probes is performed in the presence of 1-(3-dimethyl aminopropyl)-3-ethylcarbamide hydrogen chloride (EDC) and n-methylimidazole. The amino group is at the 3′ end of the probe, and thus the 3′ end is coupled to the well (the matrix) whereby any potential elongation of the probe during the amplification process is blocked. Other methods of pre-immobilizing the capture probes are readily available in the art.

In an embodiment where the solid support matrix is in form of beads, the oligonucleotide probes may be coupled to the beads in a post-synthetic manner or by in situ synthesis (see e.g., Lövgren et al. in Clin. Chem. 1997, 43(10): 1937-43 and Hakala and Lonnberg in Bioconjugate Chem. 1997, 8:232-7).

In a preferred embodiment of the present invention the amplification step is performed as an asymmetric PCR reaction carried out in microtitration wells comprising the oligonucleotide probe arrays. The amplification is per-formed in the presence of an excess of biotin labelled primers. It will, however, be apparent to a man skilled in the art, that other types of labels are equally useful in this embodiment.

In a preferred embodiment of the present invention the reagent mixture includes fluorescently labelled avidin, described by Marttila et al. in FEBS Letters, 1998, 441:313-317. The label used in the embodiment is a thermo-stable fluorescent Eu(III) chelate (W8044; Wallac). Thermo stable streptavidin has been described in Reznik et al., Nat Biotechnol, 1996, 14:1007-1011. The thermo stability is obtained by the addition of intermonomeric disulfide bridges in the tetramer.

After completed amplification, the temperature of the reaction mixture is lowered to a temperature allowing hybridization between the amplified targets and the probe arrays, for example down to 33° C. The amplified target nucleic acids, now comprising the biotin from the biotin labelled primers and detectably labelled through binding to fluorescently labelled avidin, are captured onto the matrix by means of hybridization to the capture probes. This scheme is an example of how to perform the integrated non-homogeneous method of amplifying and analysing nucleic acids according to the present invention. Other useful schemes are readily apparent to a man skilled in the art.

In this exemplified embodiment of the present method the wells are washed after the hybridization incubation (step iv) and the resulting labelled hybrids are detected by imaging, e.g., using modified time-resolved fluorescence microscopy as described by Seveus et al. in Cytometry, 1992, 13(4):329-38.

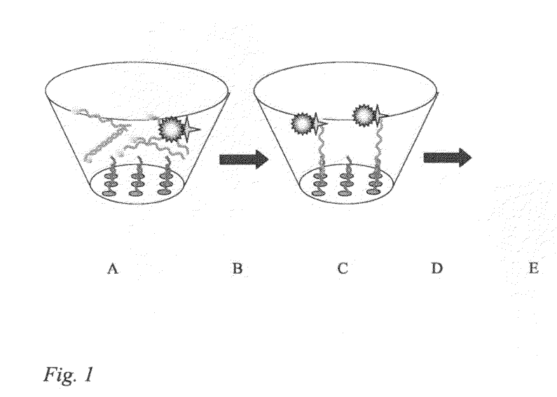

FIG. 1 pictures the principle of such an integrated amplification and detection assay performed in a microtitration well. In the figure, A represents a well comprising immobilized probes, sample DNA, reaction mixture for performing an amplification of the sample DNA and the labelled thermo stable avidin, at point B the temperature is lowered to allow hybridization (C) of the amplified sample DNA with the immobilized oligonucleotide probes. The amplification product comprises of biotin, which binds the labelled avidin. The un-bound reactants are then washed and the wells are dried (D) and then visualized and photographed by modified time-resolved fluorescence microscopy in step E.

The present invention provides a feasible way of combining amplification of DNA, such as PCR, and heterogeneous hybridization assay formats, such as DELFIA® hybridization in an integrated assay. Such an assay is achieved by coating a microtitration well with a universal probe. Allele-specific, 3′ end-blocked, labelled oligonucleotide probes (e.g. W2014 terbium(III), samarium(III) and dysprosium(III) chelate labelled probes; Wallac) are included in the reaction mixture together with an amplification control label, e.g., europium(III) chelate labelled thermo stable avidin. After performing an amplification reaction in the well, the temperature is lowered and the amplification product is allowed to hybridize with the labelled allele-specific probes and with the immobilized universal probe. After washing, the DELFIA® signal is enhanced and measured.

The advantages of an integrated method according to the present invention comprise fewer manipulations steps, making the system easily auto-mated, and lowering the risk of laboratory contamination as the amplified DNA sample is not transferred from the amplification chamber to a separate device for analysis and imaging. These advantages are easily pictured by the following chart comparing a method of performing conventional PCR followed by hybridization and detection by DELFIA® with an integrated method according to the present invention.

Further experiments showing the feasibility of the method of the pre-sent invention are given in the examples. It will be obvious to a person skilled in the art that, as the technology advances, the inventive concept can be implemented in various ways. The invention and its embodiments are not limited to the examples described above but may vary within the scope of the claims.

### EXAMPLES

### Example 1

### Integrated Amplification and an Analysis in a Solid-Phase Hybridization

This example is a comparison between conventional PCR per-formed in a tube, in a microtiter well and an integrated PCR according to the present invention. The amplification product was analysed in a solid-phase hybridization with pre-immobilized oligonucleotide probe.

First, the black NUCLEOLINK™ (Nunc) strips were coated with DBQ1 oligonucleotide probe specific for all DBQ1 alleles (5′ NH₂ modified U TTT TTT TTT TCT TCG ACA GCG AC 3′; SEQ ID NO: 1). An 1-ul aliquot of 10 mM EDAC [1-ethyl-3-(3-dimethyl-amino-propyl)carbodiimide], 10 mM methyl imidazole and 0.4 μM oligonucleotide was added onto the well and incubated at room temperature, 30 min. After immobilization, the wells were washed 6 times with DELFIA® Wash Solution (Wallac) in a DELFIAE Platewash (Wallac).

Integrated PCR was performed in NUCLEOLINK™ microtitration wells where an oligonucleotide probe had been pre-immobilized in a total volume of 10 ul, and as a reference, in standard PCR tubes in a total volume of 50 ul and in non-immobilized NUCLEOLINK™ microtitration wells in a total volume of 15 ul. In all reaction types, the PCR was performed in equal reaction mixture and cycling conditions. The reaction mixture was following: 1.5×DYNAZYME™ buffer (Finnzymes), 0.4 mM dNTP's, 3 mM MgCl₂, 0.5 M betaine, 0.025% BSA, 0.05 μM DQB1 forward primer (5′ GCT ACT TCA CCA ACG GGA C 3′; SEQ ID NO: 2), 0.2 μM biotinylated DQB1 reverse primer (5′ Biotin TTC TGG CTG TTC CAG TAC TC 3′; SEQ ID NO: 3), 0.03 U/μl DYNAZYME™ enzyme (Finnzymes) and 2 ng/μl genomic DNA sample purified from a volunteer's whole blood.

The PCR program was as follows: Preheating +95 1 min; 35 cycles of following +95° C. 1 min, +60° C. 1 min, +74° C. 1 min and final denaturation 8 min, whereafter the reaction mixture was cooled down to +4° C.

The products from integrated PCR were analyzed by adding 10 μl of streptavidin labelled with a stable fluorescent W8044 europium(III) chelate (Wallac) to the final concentration of 50 nM in DELFIA® Assay Buffer (Wallac) supplemented with 0.1% Tween 20 and 1 M NaCl onto the well and incubated at +33° C., over night. The amplification products from the reference amplifications were analysed also in a solid-phase hybridization. After amplification, 3 μl of amplification product and 17 μl of streptavidin labelled with a stable fluorescent W8044 europium (III) chelate to the final concentration of 50 nM in DEL-FIA® Assay Buffer supplemented with 0.1% Tween 20 and 1 M NaCl onto the pre-immobilized well and incubated at +33° C., 5 h. After hybridization, all wells were washed 6 times with preheated DELFIA® Wash Solution (+42° C.) in a DELFIAE Platewash.

After washing, the wells were allowed to dry and then the fluorescence was measured by VICTOR²™ Plate Fluorometer (Wallac) using standard europium program.

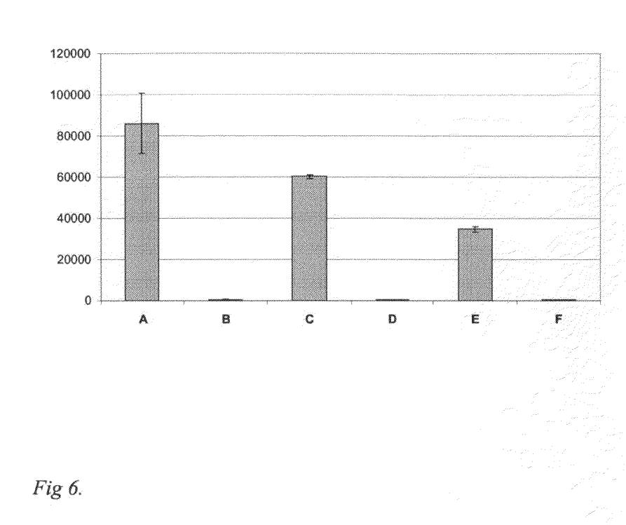

The result of this example is shown in FIG. 6 as mean fluorescence measured from 6 parallel reactions, wherein A represents the fluorescence obtained from the sample analyzed from the PCR in tube with and without sample in PCR, respectively. C and D represent the result obtained from the corresponding samples from the PCR in well, and E and F represent the fluorescence obtained from the integrated PCR with and without sample, respectively. The result clearly shows that the amplification can be performed in the hybridization well. The hybridization signal from integrated PCR is comparable to that of reference amplifications performed in separate vessels, all having high signal-to-background ratios.

### Example 2

### Integrated Amplification and Analysis of a 4-Oligo Array

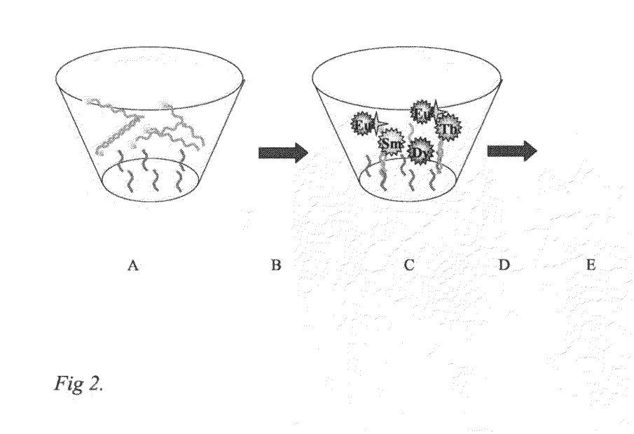

This example is a comparison between an integrated PCR consisting of a post-amplification addition of label (principle presented in FIG. 2), and a totally integrated PCR including the label according to the present invention (principle presented in FIG. 1). In this same experiment, the thermo stable avidin is compared to streptavidin. The amplification product was analysed in a solid-phase hybridization with a pre-immobilized 4-oligonucleotide array.

First, four oligonucleotide probes were spotted onto the black NU-CLIOLINK™ (Nunc) strips [DBQ1 oligonucleotide probe specific for all DBQ1 alleles (5′ NH₂ modified U TTT TTT TTT TCT TCG ACA GCG AC 3′; SEQ ID NO: 1); DQB1*0602,0603 (5′ NH₂ modified U TTT TTT TTT TGT GTA CCG CGC 3′; SEQ ID NO: 4); DQB1*0603,0604 (5′ NH₂ modified U TTT TTT TTT TGT AAC CAG ACA CA 3′; SEQ ID NO: 5); and DQB1*0201-3 (5′ NH₂ modified U TTT TTT TTT TAG AGA GAT CGT GCG 3′; SEQ ID NO: 6)]. The spotting solution consisted of 10 mM EDAC, 10 mM methyl imidazole and 3 μM oligonucleotide and the spots were printed by a spot assayer (BIOCHIP AR-RAYER™, Packard), 3 drops per spot with a spacing of 800 μm from spot to spot.

Prior to amplification, the plates were pre-washed four times with DELFIA® Wash Solution. The PCR amplification reaction was performed in the pre-immobilized wells in a total volume of 10 μl. In totally integrated PCR, the label was included in the PCR reaction mixture; in comparison, the label was excluded from the PCR mixture and added after the cycling. The reaction mixture was the following: 1.5×DYNAZYME™ buffer (Finnzymes), 0.4 mM dNTP's, 3 mM MgCl₂, 0.5 M betaine, 0.025% BSA, 0.05 μM DQB1 forward primer (5′ GCT ACT TCA CCA ACG GGA C 3′; SEQ ID NO: 2), 0.2 μM biotinylated DQB1 reverse primer (5′ Biotin TTC TGG CTG TTC CAG TAC TC 3′; SEQ ID NO: 3), 0.03 U/μl DYNAZYME™ enzyme (Finnzymes), 2 ng/μl genomic DNA sample purified from a volunteer's whole blood, and optionally 50 nM streptavidin or thermo stable avidin, labelled with a stable fluorescent W8044 europium(III) chelate.

The PCR program was as follows: Preheating +95 1 min; 30 cycles of following +95° C. 1 min, +60° C. 1 min, +74° C. 1 min and final denaturation 8 min. After amplification, in a totally integrated PCR, the reaction mixture was cooled down to hybridization temperature +33° C. and allowed to hybridize over night.

When the label was added after cycling, the reaction mixture was cooled down to +4° C. A 10-μl aliquot of the W8044 labelled streptavidin or thermo stable avidin was added to the reagent mixture in DELFIA® Assay Buffer supplemented with 0.1% Tween 20 to the final concentration of 50 nM and the hybridization reaction was incubated over night in +33° C.

Following hybridization, the wells were washed 6 times with pre-heated DELFIA® Wash Solution (+42° C.) in a DELFIA® Platewash, and dried, whereafter the result was imaged in a modified time-resolved microscopy.

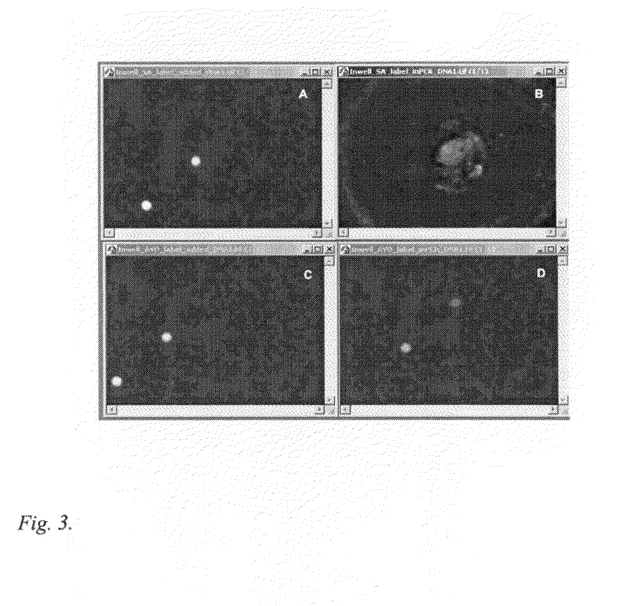

The results of this experiment are shown in FIG. 3, wherein panel A shows the result of an integrated assay using W8044 europium(III) labelled streptavidin as label added after the PCR-reaction; panel B shows the result of a totally integrated assay where the W8044 europium(III) labelled streptavidin is added in the PCR-reaction; panel C shows the result of an integrated assay using thermo stable avidin labelled with W8044 europium(III) as label added after the PCR-reaction; and panel D shows the result of a totally integrated assay where the W8044 labelled thermo stable avidin is added in the PCR-reaction. The sample analyzed had been previously genotyped to be DQB1*0201 so, as expected, the hybridization signal is detected from the DQB1 probe specific to all alleles (up right) and DQB1*0201-3 probe (down left). The figure shows that even in the totally integrated reaction where the labelled thermo stable avidin is added already in the amplification (D), the hybridization pattern is equal to those where the labelled streptavidin (A) or avidin (C) is added after the amplification. Panel B shows that the labelled streptavidin has been denatured during cycling.

### Example 3

### PCR DELFIA® Assay

In this example, the integration of amplification and heterogeneous hybridization assay formats is exemplified by combining DELFIA® hybridization and PCR in an integrated assay.

First, the black NUCLEOLINK™ (Nunc) strips were coated with DBQ1 oligonucleotide probe specific for all DBQ1 alleles (5′ NH₂ modified U TTT TTT TTT TCT TCG ACA GCG AC 3′; SEQ ID NO: 1). An 20-ul aliquot of 10 mM EDAC [1-ethyl-3-(3-dimethyl-amino-propyl)carbodiimide], 10 mM methyl imidazole and 0.2 μM oligonucleotide was added onto the well and incubated over night at room temperature. After immobilization, the wells were washed 3 times with DELFIA® Wash Solution and 3 times with water in a DELFIA® platewash.

The PCR amplification reaction was performed in the pre-immobilized wells in the given reaction mixture (10 μl): 1.5×DYNAZYME™ buffer (Finnzymes), 0.4 mM dNTP's, 3 mM MgCl₂, 0.5 M betaine, 0.025% BSA, 0.05 μM DQB1 forward primer (5′ GCT ACT TCA CCA ACG GGA C 3′; SEQ ID NO: 2), 0.2 μM biotinylated DQB1 reverse primer (5′ Biotin TTC TGG CTG TTC CAG TAC TC 3′; SEQ ID NO: 3), 0.03 U/μl DYNAZYME™ enzyme (Finnzymes) and 2 ng/μl genomic DNA sample purified from a volunteer's whole blood.

The PCR program was as follows: Preheating +95 1 min; 35 cycles of following +95° C. 1 min, +60° C. 1 min, +74° C. 1 min and final denaturation 8 min, whereafter the reaction mixture was cooled down to +4° C.

The products from integrated PCR were analyzed by adding 10 μl of probe solution onto the PCR well. The probe solution consisted of 200 nM streptavidin labelled with W8044 europium(III) chelate, 0.2 ng/μl DQB1*06 specific oligonucleotide probe labelled in its NH₂ moiety with W2014 samarium(III) chelate (Wallac) [5′ (NH₂ modified U)₂₀ TTG TAA CCA GAC ACA; SEQ ID NO: 7], 0.1 ng/μl DQB1*02 specific oligonucleotide probe labelled with W2014 terbium(III) chelate (Wallac) [5′ (NH₂ modified U)₂₀ GAA GAG ATC GTG CG; SEQ ID NO: 8] in 100 mM Tris-HCl pH 7.5, supplemented with 10% PEG, 1 M NaCl, 0.1% Tween 20 and 250 μM EDTA. The amplified sample DNA was allowed to hybridize with the pre-immobilized capture probe and the labelled oligonucleotide probes for 3 h in +33° C. As amplification control, the labelled streptavidin was bound to the target comprising biotin. After hybridization, the wells were washed 6 times with preheated DELFIA® Wash Solution (+42° C.) in a DELFIA® Platewash.

For detection, 200 μl of DELFIA® Enhancement Solution (Wallac) per well were added, and the wells were incubated at room temperature for 15 min in shaking. The europium and samarium fluorescence was measured by VICTOR²™ Plate Fluorometer using standard europium/samarium program. After Eu and Sm measurement, 50 μl of DELFIA® Enhancer (Wallac) was added and after brief shaking, the terbium fluorescence was measured.

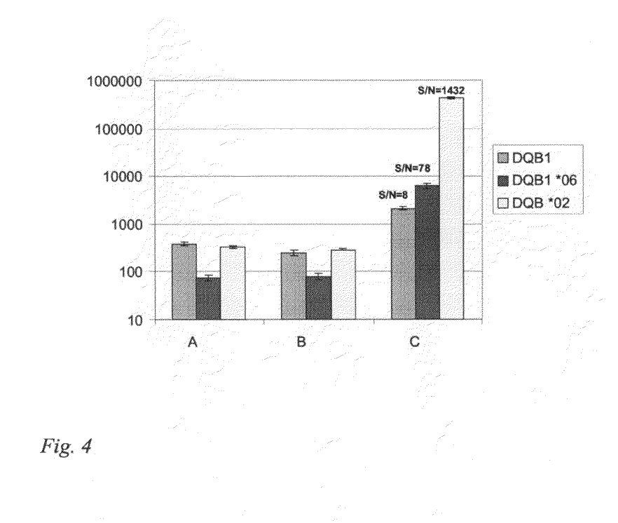

The results of this experiment are shown in FIG. 4, wherein A represents the negative hybridization control where the labelled probes have been incubated in the pre-immobilized well without sample. B represents negative PCR control where the integrated PCR has been performed in the pre-immobilized wells without sample and thereafter hybridized with the labelled probes. C represents the DNA sample analyzed in the integrated PCR. The sample analyzed had been previously genotyped to be DQB1*0201/0603. In the graph, DQB1 bar represents the fluorescence obtained in europium measurement from the europium labelled streptavidin control which is bound to all amplicons, DQB1*06 bar represents the fluorescence obtained in samarium measurement from the DQB1*06 specific probe, and DQB1*02 represents the represents the fluorescence obtained in terbium measurement from the DQB1*02 specific probe. The negative controls show only little signal as the sample amplified and analyzed in the integrated PCR shows high signal-to-noise ratios with the allele-specific probes and control, as expected.

### Example 4

### Integrated Amplification and Analysis of a Saliva Sample

This example shows that a totally integrated PCR including the label according to the present invention can be also used to analyze crude samples. A dried saliva sample is first eluted and then amplified in a well comprising a model array of oligonucleotide probes.

Sample preparation: Saliva sample from a volunteer was spotted onto ISOCODE® collection paper (Schleicher&Schuell) and onto FTA® collection paper (Whatman), and 1.5 mm disks punched from the dried spot. The disks were rinsed in 500 μl of H20 and the supernatant was removed, whereafter the disks were heated in 50 μl of H20 in 95° C. for 30 min. The supernatant was subsequently retrieved and used as a sample.

First, an oligonucleotide probe was spotted onto the black NUCLEOLINK™ (Nunc) strips in 9 positions [DBQ1 oligonucleotide probe specific for all DBQ1 alleles (5′ NH₂ modified U TTT TTT TTT TCT TCG ACA GCG AC 3′; SEQ ID NO: 1)]. The spotting solution consisted of 10 mM EDAC, 10 mM methyl imidazole and 3 μM oligonucleotide and the spots were printed by a spot assayer (BIOCHIP ARRAYER™, Packard), 3 drops per spot with a spacing of 500 μm from spot to spot.

Prior to amplification, the plates were pre-washed four times with DELFIA® Wash Solution. The integrated amplification reaction was performed in the pre-immobilized wells in a total volume of 20 μl of following reaction mixture: 1.5×DYNAZYME™ buffer (Finnzymes), 0.4 mM dNTP's, 3 mM MgCl₂, 0.5 M betaine, 0.025% BSA, 0.05 μM DQB1 forward primer (5′ GCT ACT TCA CCA ACG GGA C 3′; SEQ ID NO: 2), 0.2 μM biotinylated DQB1 reverse primer (5′ Biotin TTC TGG CTG TTC CAG TAC TC 3′; SEQ ID NO: 3), 0.04 U/μl DYNAZYME™ enzyme (Finnzymes), 50 nM thermo stable avidin, labelled with a stable fluorescent W8044 europium(III) chelate, 10 μl of disk eluent (ISOCODE® or FTA® eluent) or as a reference, 2 ng/μl genomic DNA sample purified from a volunteer's whole blood.

The PCR program was as follows: 8 cycles of preheating +4 C 30 sec, +95 3 min; 30 cycles of following +95° C. 1 min, +60° C. 1 min, +74° C. 1 min and final denaturation 8 min. After amplification, in a totally integrated PCR, the reaction mixture was cooled down briefly to +4° C. and directly to hybridization temperature +33° C. and allowed to hybridize over night.

Following hybridization, the disk was removed from the wells and the wells were washed 6 times with preheated DELFIA® Wash Solution (+42° C.) in a DELFIA® Platewash, and dried, whereafter the result was imaged in a modified time-resolved microscopy.

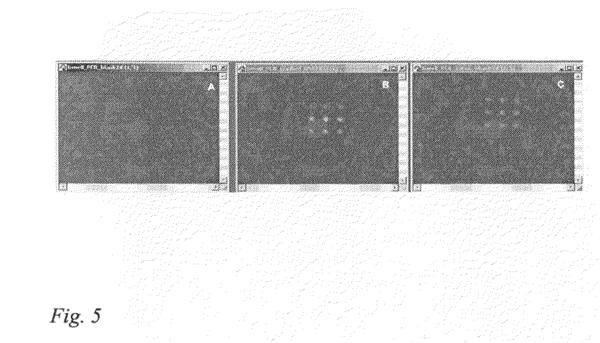

The results of this experiment are shown in FIG. 5, wherein panel A shows the result of a PCR control without sample DNA; panel B shows the result of a positive control using purified DNA as sample; and panel C shows the result of an assay using saliva eluent derived from the ISOCODE® collection paper. According to manufactures, the 1.5-mm saliva disks contain typically 0.3-70 ng of DNA of which one fifth was used in the integrated PCR in this experiment. The figure proves that the method according to present invention is sensitive enough to analyze limited amounts of sample DNA in crude samples.

1. A method of amplifying and analysing target nucleic acids, comprising the following steps:
i) providing oligonucleotide capture probes immobilized or immobilizable on a solid support;
ii) adding a sample containing target nucleic acids and a reagent mixture, containing necessary reagents for amplification and subsequent analysing of said target nucleic acids;
iii) amplifying said target nucleic acids;
iv) hybridizing said amplified target nucleic acids to the capture probes and immobilizing said probes, if necessary;
v) separating the hybrids formed in step (iv) from un-reacted components;
vi) detecting and measuring the amount of hybridized target nucleic acids by means of a detectable signal, wherein
steps (i) to (vi) are performed in one reaction chamber.

2. The method according to claim 1, wherein said solid support is a heat stable polymer selected from the group comprising polypropylene, polycarbonate, silica and glass.

3. The method according to claims 2, wherein said polymer is functionalised.

4. The method according to claim 3, wherein said solid support is a multi-well microtitration plates or a bead.

5. The method according to claim 4, wherein said amplification in step (iii) is an amplification reaction involving thermo cycling.

6. The method according to claim 5, wherein reagents are thermo stable.

7. The method according to claim 1, wherein said sample containing target nucleic acids is a biological sample selected from the group comprising bodily fluids such as blood, saliva, sputum, urine, peritoneal and pleural fluids, lavations such as bronchoalveolar, nasal, cervical and intestinal samples, aspiration samples, biopsy samples, cell cultures, microbial cultures, prepurified DNA samples, prepurified RNA samples, samples derived from food and environmental samples.

8. The method according to claim 1, wherein said hybridization step iv) is performed after step iii) by lowering the reaction temperature to a level allowing hybridization.

9. The method according to claim 1, wherein said separation step v) is performed by adding and removing a wash solution.

10. The method according to claim 9, wherein said un-reacted components and said wash solution is removed by filtration or suction.

11. The method according to claim 1, wherein said separation step is performed by optical separation.

12. The method according to claim 11, wherein said detectable signal is detected by confocal detection.

13. The method according to claim 12, wherein said detection is performed from underneath the reaction chamber.

14. A reaction mixture for use in a method according to claim 1, comprising:
i) oligonucleotide primers labelled with a thermo-stable first member of a biorecognition pair;
ii) dNTP's;
iii) at least one polymerase;
iv) amplification and hybridization buffers;
v) a detectably labelled second member of the biorecognition pair of reagent (i).

15. The reaction mixture according to claim 14, further comprising an array of beads for immobilization of the hybrids formed in step iv).

16. The reaction mixture according to claims 14 claim 15, wherein said first member of a biorecognintion pair is biotin and said second member of the biorecognition pair is thermo stable avidin or steptavidin.

17. A kit for use in a method according to claim 13, comprising:
i) oligonucleotide primers labelled with a thermo-stable first member of a biorecognition pair;
ii) dNTP's;
iii) at least one polymerase;
iv) amplification and hybridization buffers;
v) a detectable labelled second member of the biorecognition pair of reagent (i), and
pre-immobilized target specific oligonucleotide probes.

18. The kit according to claim 17, further comprising a multi-well reaction chamber.

19. The kit according to claim 18, further comprising an array of beads for immobilization of the hybrids formed in step iv) of the method.

20. A reaction mixture for use in a method according to claim 13, comprising:
i) oligonucleotide primers labelled with a thermo-stable first member of a biorecognition pair;
ii) dNTP's;
iii) at least one polymerase;
iv) amplification and hybridization buffers;
v) a detectably labelled second member of the biorecognition pair of reagent (i).

21. The reaction mixture according to claim 14, wherein said first member of a biorecognintion pair is biotin and said second member of the biorecognition pair is thermo stable avidin or steptavidin.

22. A kit for use in a method according to claim 1, comprising:
i) oligonucleotide primers labelled with a thermo-stable first member of a biorecognition pair;
ii) dNTP's;
iii) at least one polymerase;
iv) amplification and hybridization buffers;
v) a detectably labelled second member of the biorecognition pair of reagent (i), and
pre-immobilized target specific oligonucleotide probes.


SEQUENCE LISTING (8 sequences)

SEQ ID NO: 1 - DNA, 23 bp, Artificial
  Probe
ntttttttttcttcgacagcgac

SEQ ID NO: 2 - DNA, 19 bp, Artificial
  Primer
gctacttcaccaacgggac

SEQ ID NO: 3 - DNA, 20 bp, Artificial
  Primer
ttctggctgttccagtactc

SEQ ID NO: 4 - DNA, 22 bp, Artificial
  Probe
nttttttttttgtgtaccgcgc

SEQ ID NO: 5 - DNA, 24 bp, Artificial
  Probe
nttttttttttgtaaccagacaca

SEQ ID NO: 6 - DNA, 25 bp, Artificial
  Probe
nttttttttttagagagatcgtgcg

SEQ ID NO: 7 - DNA, 35 bp, Artificial
  Probe
nnnnnnnnnnnnnnnnnnnnttgtaaccagacaca

SEQ ID NO: 8 - DNA, 34 bp, Artificial
  Probe
nnnnnnnnnnnnnnnnnnnngaagagatcgtgcg